(19) United States
(12) Patent Application Publication
(10) Pub. No.: US 2026/0326127 A1
(43) Pub. Date: Oct. 22, 2026

(54) DEVICE FOR SPRAYING OZONATED WATER IN AN AGRICULTURAL ENVIRONMENT

(71) Applicants: ECOLE D'INGENIEURS DE PURPAN, TOULOUSE (FR); INSTITUT NATIONAL DES SCIENCES APPLIQUEES DE TOULOUSE, TOULOUSE (FR); CENTRE NATIONAL DE LA RECHERCHE SCIENTIFIQUE, PARIS (FR); INSTITUT NATIONAL DE RECHERCHE POUR L'AGRICULTURE, L'ALIMENTATION ET L'ENVIRONNEMENT, PARIS (FR); INSTITUT NATIONAL POLYTECHNIQUE DE TOULOUSE, TOULOUSE (FR)

(72) Inventors: Axel CANADO, TOULOUSE (FR); Gilles HEBRARD, TOULOUSE (FR); Marielle PAGES; Frédéric VIOLLEAU, TOULOUSE (FR); Nicolas DIETRICH, TOULOUSE (FR)

(21) Appl. No.: 19/110,592

(22) PCT Filed: Sep. 15, 2023
(86) PCT No.: PCT/FR2023/051412

§ 371 Date: Mar. 11, 2025

(30) Foreign Application Priority Data
Sep. 15, 2022 (FR) 2209282

(51) Int. Cl. B05B 7/26; A01M 7/00; A61L 2/22; A61L 103/05

(52) U.S. Cl. CPC B05B 7/262; A01M 7/005; A61L 2/22; A61L 2103/05; A61L 2202/15

(57) ABSTRACT

A device for spraying ozonated water in an agricultural environment has a collar defining a dispensing opening (4) for dispensing both water and ozone together. The water is sprayed by a nozzle (10) set back from the opening. The nozzle is supplied from a tank (7) installed with the device. The ozone gas is dispensed via a gas inlet (22) connected to a device for producing and/or storing gas (9, 13) which is also installed with the device. The gas inlet is also set back in the collar such that the collar defines a chamber (28, 29) for confining the ozone gas emitted around the sprayed liquid in order to allow the sprayed liquid to be enriched with dissolved ozone.

### TECHNICAL FIELD

The present invention relates to a spraying device, in particular intended to be used in the open air, outdoors, in particular in an agricultural environment, on hectares of crops to be treated, or indoors. The advantage of the invention is that it is possible to spray a liquid laden with gas or gaseous solute, while at the same time limiting the desorption of the gaseous solute or gas from the sprayed liquid. This device can more particularly spray ozone-laden water via a nozzle, in order to carry out a treatment, whether of the phytosanitary type for crops or of the chemical type for an action on a surface to be treated.

### TECHNOLOGICAL BACKGROUND

In the prior art, there are known ozonated water diffusion devices for sterilization, disinfection and deodorization in an industrial setting and in closed environments with controlled atmosphere. In particular, documents U.S. Pat. Nos. 5,431,861, 6,076,748 or else JP2008229491 describe such devices. Document JP2008229491 provides for the introduction of outside air through orifices made in the convergence means by virtue of the negative pressure generated during spraying.

On an experimental scale, ozonated water is known for its effectiveness in combating the development of certain pathogens. Ozonated water could be an alternative to phytosanitary treatments based on synthetic products from the chemical industry. Specifically, ozonated water has a major ecological advantage in that the remanence of the ozone molecule is very low, and decomposes into environmentally neutral dioxygen.

However, since the half-life of gaseous ozone in air at 20° C. is 3 days, while the half-life of ozone dissolved in water at 20° C. is 20 min, ozonated water has never been able to be used effectively in agricultural environments to carry out phytosanitary treatments. Specifically, in the open air, it is not possible to force the gaseous ozone to remain in contact with the crops to be treated, because of climatic uncertainty and wind. In addition, the solubilization of ozone in the aqueous phase reduces the half-life time of ozone to a duration of the order of 20 minutes at ambient temperature and remains problematic. Specifically, spraying ozonated water through a nozzle in the direction of a target to be treated is associated with a strong desorption of ozone in the sprayed droplets, making this action ineffective.

### SUMMARY OF THE INVENTION

The object of the invention is to propose a solution that is adaptable to the agricultural environment and of which the efficiency is demonstrated in the phytosanitary treatment of the plants in question. The advantage of the invention is that is proposes a technical solution that makes it possible to maintain an effective concentration of ozone in a sprayed liquid either by promoting the absorption of ozone in this liquid, and/or by limiting its desorption.

The advantage of the invention is that it makes it possible to spray a liquid in a gaseous environment, in order to increase the absorption of the gaseous solute or to limit the desorption thereof. These spraying conditions correspond to a difference in speed between the ejected liquid and the gas diffused around the ejected liquid, causing friction to occur at the surface of the ejected liquid. This friction induces the transformation of the jet of liquid in the form of a liquid film into ribbons, then into ligaments that then break into relatively spherical particles in order to produce a cloud of drops.

The invention consists in promoting the dissolution of the gas in the form of a gaseous solute in the ejected liquid, in particular at the moment when the liquid is still predominantly in the form of a jet, film or ligaments, and before the majority of the liquid is in the form of drops. The invention also aims to limit desorption of the gaseous solute (ozone) that is already dissolved in the ejected liquid, in particular when the liquid is still predominantly in the form of a jet, film or ligament. The invention also consists in limiting the presence of gas other than that of the gaseous solute around the liquid, when the liquid is still predominantly in the form of a jet, film or ligaments. The invention promotes the presence of gas around the liquid sprayed in the form of a jet, film or ligaments, in order to increase the proportion of gaseous solute of this gas in the liquid.

One subject of the invention is a spraying device, in particular for spraying ozonated water in an agricultural environment, the device having
an opening for jointly dispensing liquid and gas,
a nozzle intended to be connected to a reservoir of liquid to be sprayed,
a gas inlet of a device for producing and/or storing gas,
   and a collar surrounding the nozzle and the gas inlet such that an edge of this collar defines the dispensing opening of the device, and such that a spraying orifice of the nozzle is at a distance (dr) set back in the collar relative to this dispensing opening, the collar defining a chamber for confining the gas emitted through the gas inlet, the confinement chamber extending between the spraying orifice and the dispensing opening. In other words, the collar defines a channeling device. This channeling device makes it possible in particular to avoid the desorption of ozone from the dispensed liquid.

In particular, a difference in speed of emission of the liquid through the spraying orifice is between 50 and 1500 times, preferably between 100 and 300 times, greater than a speed of dispensing of the gas through the gas inlet, such that the liquid can be predominantly in the form of a jet, film or ligaments in the collar. In particular, a dispensing speed of the liquid is of the order of 5 to 15 m/s while a dispensing speed of the gas is of the order of 1 to 10 cm/s.

Preferentially, the spraying orifice can dispense the liquid according to a spray distributed spatially in at least one plane such that a width of the opening measured in this plane and orthogonal to a spraying axis Y of the nozzle represents between 80% and 140%, and more particularly between 95% and 120%, of a spray width in this same plane for a spray produced by said nozzle without its collar, this spray width being measured at a distance from the spraying orifice that is equal to the set-back distance.

More particularly, the spraying orifice can dispense the liquid according to a spray distributed spatially in at least one plane and having a spray thickness transverse to this plane such that a thickness of the opening measured transverse to this plane represents more than 100% and less than 140% of the spray thickness transverse to this same plane for a spray produced by said nozzle without its collar, the spray thickness being measured at a distance from the spraying orifice that is equal to the set-back distance.

Advantageously, the spraying orifice can dispense the liquid according to a spray having a portion in the form of liquid film having a continuous surface such that a length of the continuous surface of this film is measured along a spraying axis of the nozzle represents between 40 and 120% of the set-back distance.

In particular, the spraying orifice may be in the form of a longitudinal slot along an elongation axis Z, and in this case the dispensing opening preferentially has a main elongation axis parallel to the axis of the slot.

In particular, the gas inlet may be disposed set back from the spraying orifice relative to the dispensing opening. Such a configuration makes it possible to avoid the formation of turbulence at the spraying orifice, the gas then being able to be dispensed into an annular volume and diffused annularly around the nozzle.

In particular, for homogeneous diffusion of the gas in the confinement chamber, the device may have at least two gas inlets that are evenly distributed, i.e. diametrically opposite one another when there are only two inlets, and for example supplied with an identical gas sourced through one and the same device for producing and/or storing gas.

Advantageously, the nozzle may be central and the gas inlet lateral in the collar (20), more particularly the gas inlet may be adjacent to an inner wall of the collar.

This configuration allows central diffusion of the liquid, and constrains the flows of the streams of gas against the wall of the collar. When the wall is continuous, and has no lateral opening, the gas is therefore diffused collinearly to the stream of liquid emitted by the nozzle.

Thus, the mixture of gas and liquid is diffused inside the collar, collinearly, and over a minimum distance of 1 cm, better still 3 cm, and even better still 5 cm, from the dispensing orifice of the nozzle, and inside said collar.

Preferably, the volume of the confinement chamber may be parallelepipedal. Advantageously, a transverse section of the confinement chamber, in particular close to the spraying orifice, has a section that is of substantially identical surface area to the section of the dispensing opening.

The advantage of the invention is that it proposes a collar that makes it possible to define a confinement chamber. De facto, the configuration of the nozzle and the collar are jointly determined such that the spraying orifice sprays a spray of which the nominal volume of the envelope surface of this spray, inside the collar, represents a volume less than 5% of the internal volume of the collar.

Advantageously, the dispensing opening is a single opening, and the collar is formed by a continuous wall, such that all the gases and liquids contained in the confinement chamber can be controlled. The existence of this continuous wall, a continuous wall in this case having no lateral orifices, makes it possible to limit the presence of ambient air close to the spraying orifice. Thus, in the absence of lateral openings or orifices in the wall, the wall of this collar laterally delimits the confinement chamber, the only outlet of which is then the dispensing orifice.

Another subject of the invention is a method for designing a spraying device, wherein
a nozzle is selected that is able to emit a spray of liquid distributed spatially according to an opening angle in a plane and a radial thickness transverse to this plane,
a flow rate of liquid from the nozzle is determined, in order to determine a gas flow rate as a function of this liquid flow rate,
a collar depth is determined as a function of the opening angle and the radial thickness of the spray of the nozzle.

Another subject of the invention is an assembly for treatment using a solute dissolved in a liquid, having a spraying device according to the invention, a reservoir of liquid to be sprayed, one and the same device for producing and/or storing the gas of interest that is to be diffused jointly with the liquid through the spraying device, the device for producing and/or storing gas having a diversion toward the reservoir so as to enrich the liquid contained in the reservoir with gaseous solute, such that the liquid can have a non-zero proportion of dissolved gaseous solute before the spraying thereof.

### BRIEF DESCRIPTION OF THE FIGURES

The following description with reference to the appended drawings, which are given by way of non-limiting examples, will give a good understanding of what constitutes the invention and how it can be implemented. In the appended figures:

FIG. 1 shows a diagram of agricultural spraying of a phytosanitary treatment onto a vine, in which spraying through a cloud of drops is schematically shown, and a proportion of gas in this drop cloud is also schematically depicted by a density of black dots in each drop;

FIG. 2a shows a first device for modeling dispensing of a liquid having a proportion of dissolved gas;

FIG. 2b shows a graph with measured proportions of dissolved gas in the liquid dispensed by a device according to FIG. 2a, as a function of a distance between the measuring point and an orifice for spraying this liquid through a nozzle of the device according to FIG. 2a;

FIG. 3a shows a view of a spray diffused through a nozzle of a spraying device according to the invention

FIG. 3b shows a view of the spray in FIG. 3a from a viewing angle perpendicular to that in FIG. 3a;

FIG. 4a shows a second device for modeling dispensing of a liquid having a proportion of dissolved gas according to a spraying device according to the invention;

FIG. 4b shows a graph with measured proportions of dissolved gas in the liquid dispensed by a device according to FIG. 4a, as a function of a distance between the measuring point and an orifice for spraying this liquid through a nozzle of the device according to FIG. 4a;

FIG. 5 shows a perspective view of a spraying device according to the invention;

FIG. 6 shows a view in longitudinal cross section of a spraying device according to FIG. 5;

FIG. 7 shows a perspective view of a spraying device according to a variant embodiment of the invention;

FIG. 8 shows a view in longitudinal cross section of a spraying device according to FIG. 7;

FIG. 9 shows a perspective view according to a partial longitudinal cross section of a spraying device according to FIG. 7.

### DESCRIPTION OF EMBODIMENT(S)

In the figures, and unless otherwise specified, identical elements will bear the same reference signs.

In the agricultural environment, as shown in FIG. 1, an agricultural sprayer 1 is generally a mobile device. Since crops are organized to make the mechanization of agricultural work easier, the agricultural sprayer 1 is provided with means 2 for movement, such as wheels 2 in the example in FIG. 1, in order to make it easier to move the sprayer along a plantation 3 to be treated. In particular, the agricultural sprayer 1 is towed along furrows each bearing plantations, such as vines, to be treated. The agricultural sprayer 1 is intended to disperse a phytosanitary treatment, preferably in a homogeneous dose during its movement along the crops.

In order to preserve the crops, the sprayer is kept at a distance from these crops during its movement, such that a distance “d” between a dispensing opening 4 of the device 1 and a foot 5 of the plantation 3, measured transverse to the axis of movement of the device 1, is preferably greater than the value d minus half of an average theoretical width Ith of the plantation 3, this theoretical width being measured transverse to the axis of movement of the device 1. A dispensing axis Y of the sprayer 1 is oriented perpendicular to a direction of travel X of this sprayer 1. The spraying is lateral to the movement.

In order to ensure the homogeneity of deposition of the phytosanitary treatment, the agricultural sprayer is configured to ensure the formation of a cloud of drops 6 of which the drops will mainly be propelled to a distance of which the value will be between [d−½*Iₜₕ; d+½*Iₜₕ]. At this distance, the lateral propulsion force of the drops is canceled out under the weight of these drops, which will be deposited by gravity onto the plantation 3. The agricultural sprayer 1 may have means for orienting the dispensing opening 4 such that the dispensing axis Y may be modified, and not necessarily be oriented parallel to the ground on which the wheels bear.

The sprayer 1 is intended to allow joint dispensing of liquid and gas, and also of gas dissolved in the liquid. The sprayer 1 has a first means of connection to a liquid reservoir 7. This liquid reservoir 7 can be supported by a chassis 8 on which the wheels 2 are mounted. The sprayer 1 has a second connection means so as to be connected to device for producing and/or storing gas 9, this device possibly being a gas cylinder, in which the gas is compressed. The device for producing and/or storing gas 9 is also borne by the chassis 8.

The liquid reservoir 7 contains water, or ozonated water having a non-zero proportion of ozone. The gas cylinder 9 contains gaseous ozone. Under ambient conditions, ozone tends to decompose naturally into dioxygen.

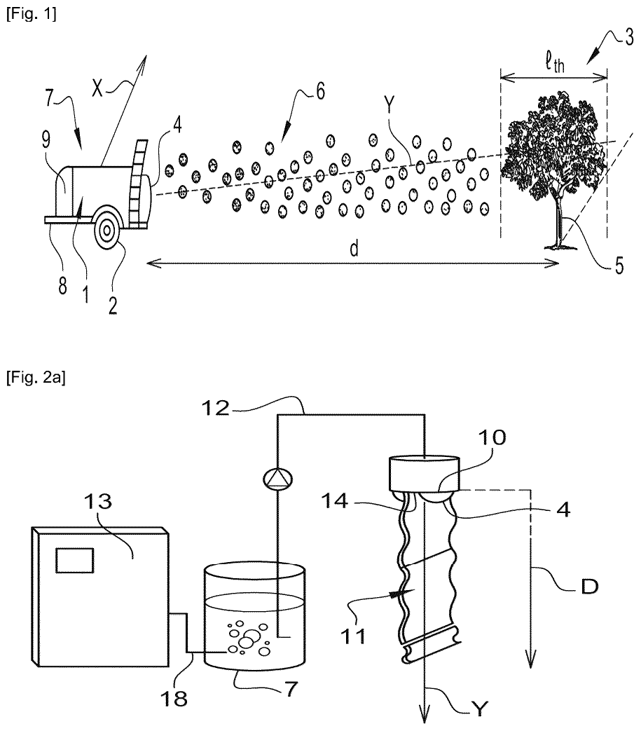

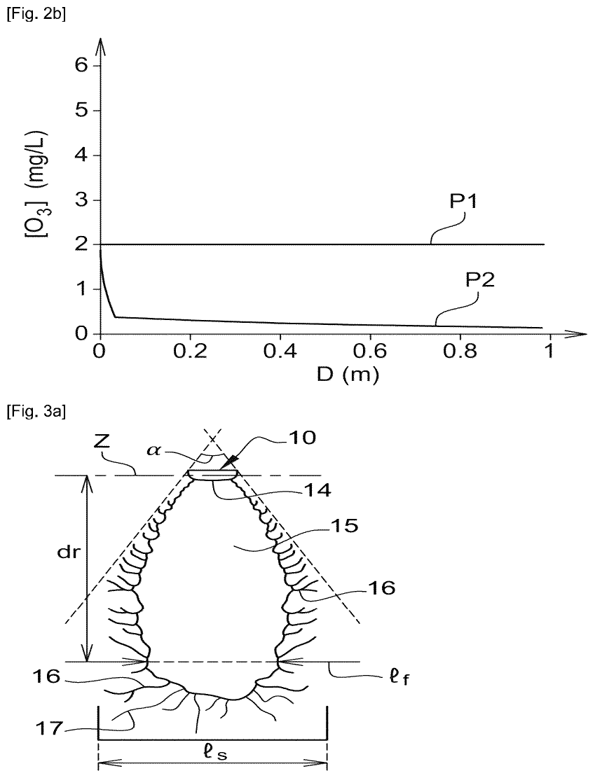

FIG. 2a shows a device for modeling a proportion of ozone (O3) in a liquid film 11 sprayed by a device for preparing ozonated water and spraying through a nozzle 10, the ozone already being dissolved in the liquid intended to be sprayed. In this model, the nozzle 10 is supplied through a means 12 for connection to a reservoir 7 of liquid, in particular water. This tank 7 is itself supplied through a pipe 18 with gas provided by an ozone generator 13. The ozone generator 13 can continuously enrich the liquid with ozone, in order to control and maintain a proportion P1 of dissolved ozone in the liquid to be sprayed through the nozzle.

FIG. 2b represents a change in the proportion P2 of dissolved ozone in the film 11, this proportion P2 is a function of a distance D away from the measurement relative to the nozzle 10 along a dispensing axis Y. This model confirms the observations concerning the inefficiency of a treatment with sprayed ozonated water insofar as, from the first centimeters at the outlet of a spraying orifice 14 of the nozzle 10, a very clear decrease in the proportion of dissolved ozone is observed in the film 11. In addition, at the distance D of 1 meter, the proportion P2 of dissolved ozone in the dispensed liquid is divided by 20 compared with the proportion P1 of dissolved ozone in the liquid of the reservoir 7.

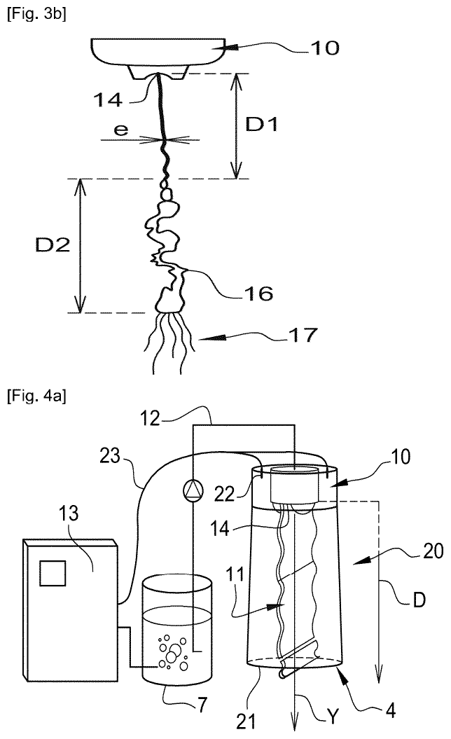

Preferably, in order to obtain a film, the nozzle 10 has a spraying orifice 14 in the form of a longitudinal slot along an axis Z, such that the jet is in the form of a film of which the contours projected onto a plane containing the axis Z has a continuous surface 15 issuing from the slot of the spraying orifice, this continuous surface 15 being bordered by filaments 16 that end in drops 17. The continuous surface 15 forms a liquid film 15. As shown in FIG. 3b, which is a view perpendicular to that in FIG. 3a, it can be seen that the continuous surface 15 is substantially flat over a distance D1 measured from the spraying orifice 14, and then undulates over a distance D2, so as to be predominantly in the state of drops 17 beyond the distance D2.

The cloud of drops is thus formed at a cumulative distance of D1 and D2 from the spraying orifice 14. There is thus a liquid film that is continuous over a cumulative distance of D1 and D2. Over the distance D1, a thickness “e” of the film is shown in FIG. 3b. This thickness e decreases as the distance from the spraying orifice 14 at which the thickness is measured increases. The farther from the spraying orifice the measurement is taken, the smaller the thickness of the film. Specifically, the continuous surface 15 tapers until it forms the filaments 16 and the drops 17.

The envelope surface of the spray that is sprayed by the spraying orifice 14 encompasses the spatial environment in which the continuous surface 15, the filaments 16 and the drops 17 are at the outlet of the spraying orifice. As a function of the spraying direction Y relative to the ground, the shape of this envelope surface changes under the effect of the forces of gravity that are applied to the drops.

In FIG. 3a, the spraying orifice dispenses the liquid according to a spray having an opening angle α. The apex of the opening angle α is situated upstream of the spraying orifice 14. In light of the observation that, from the first centimeters at the outlet of the spraying orifice 14 of the nozzle 10, a very clear decrease in the proportion of dissolved ozone was observed in the film 11, the inventors have demonstrated that this phenomenon can be contained, and at least limited by the presence of a collar 20 around the sprayed liquid, this collar being disposed around the sprayed liquid from the first centimeters around the outlet of the spraying orifice.

FIG. 4a shows a model of a spraying device according to the invention. According to the invention, the spraying device 1, also called a sprayer, has a nozzle 10 intended to be connected to a reservoir 7 by a connection 12. The nozzle is intended to dispense a liquid contained in the reservoir 7 through a spraying orifice 14. In particular, according to one embodiment, the liquid is sprayed in the form of a film 11. In the invention, the device has a collar 20 that surrounds the nozzle 10 and the spraying orifice 14 such that an edge 21 of this collar 20 delimits an opening 4 of the spray device.

According to the invention, the sprayer according to the invention 1 also has a gas inlet 22, this inlet 22 being connected to a pipe 23 so as to be connected to a gas generator 13 or a gas cylinder 9. Preferably, the gas is compressed in the gas cylinder 9. The gas inlet 22 is disposed inside the volume delimited by the collar 20.

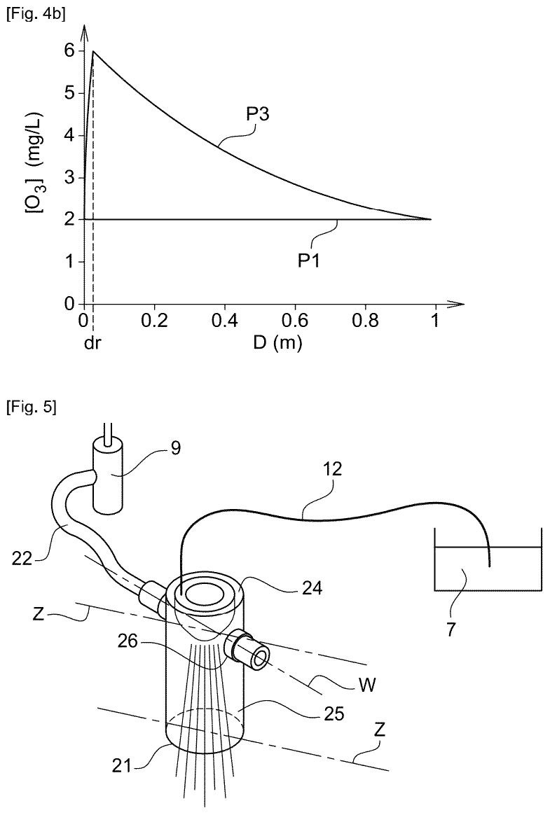

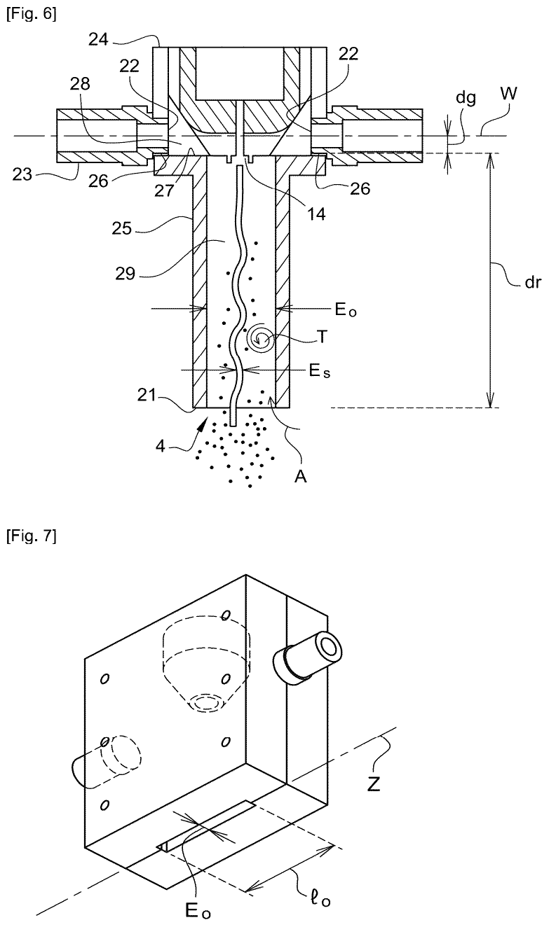

The collar 20 has a base 24 through which the body of the nozzle 10 passes, and an upright lateral wall 25 around the base 24. The lateral wall 25 has a bore 26 for receiving the gas inlet 22. As shown in FIG. 5, the collar has two bores such as 26 for receiving two gas inlets such as 22. The two gas inlets 22 are diametrically opposite one another and are oriented such that their respective streams are oriented along the same dispensing axis W. The axis W of dispensing of the gas into the collar 20 is secant with the body of the nozzle 10, as shown in FIG. 6. According to the embodiment in FIG. 6, the two gas inlets 22 supply the collar 20 with gas upstream of the spraying orifice 14, such that the gas dispensed is situated around the nozzle body and surrounds the spray sprayed through the spraying orifice 14. In this configuration, a distance dg by which the gas inlets 22 are set back relative to the opening 4 is greater than a distance between the spraying orifice and the opening. Thus, the spraying orifice is downstream of the gas inlets in the collar 20.

The nozzle body is cylindrical, the bottom 24 and the lateral wall 25 are formed so as to define an annular volume around the nozzle body. The lateral wall has an internal annular restriction 27 such that the internal volume delimited in the collar comprises the annular volume 28 around the nozzle body and a cylindrical volume 29, in fluidic continuity with the annular volume 28. The spraying orifice 14 is designed to emerge centrally, such that the spraying orifice 14 is at any point equidistant from the internal annular restriction 27. The spraying axis Y of the spraying orifice 14 is substantially parallel to the main elongation axis of the collar 20.

Inside the collar 20, there is therefore dispensing of a spray of liquid through the dispensing orifice 14 and of gas through the one or more gas inlets 22. The device therefore allows joint dispensing of liquid and gas. In addition, as has been demonstrated experimentally, the presence of the collar contributes to a very significant improvement, as shown in FIG. 4b, in the proportion of dissolved ozone in the liquid sprayed with a device according to the invention. Specifically, the presence of gaseous ozone in the first centimeters at the outlet of the dispensing orifice contributes to a very significant increase in dissolved ozone in the liquid that is sprayed. The collar contributes to the formation of a chamber for confining the gaseous ozone around the liquid spray, and better still around the film of this spray.

The spraying orifice is at a distance “dr” set back from the opening 4. In addition, as is shown schematically, turbulence T is observed in the gaseous ozone as a result of a phenomenon of aspiration of air A at the dispensing opening 4. The set-back distance is of the order of a few centimeters, for example between 4 and 10 cm.

This confinement chamber makes it possible to triple the proportion P3 of dissolved gas in the sprayed liquid compared with a reference value P1 that corresponds to the proportion of dissolved gas in the liquid before spraying. Another advantage of the device according to the invention is that it allows strong enrichment with dissolved ozone, and limitation of the phenomenon of desorption of the dissolved ozone in the sprayed liquid. By virtue of the invention, at a distance D of 1 meter from the opening, this proportion P3 of dissolved ozone is equal to that of the liquid in the reservoir. This result makes it possible to improve the efficiency of phytosanitary treatments based on ozonated water thus dispensed.

A device according to the invention, with a collar, demonstrates an effectiveness 20 times greater than that of a simple device.

In particular, this confinement chamber comprises the annular volume 28 and the frustoconical volume 29 minus the space taken up by the sprayed liquid. At a minimum, the confinement chamber extends between the spraying orifice 14 and the opening 4. Specifically, in variant embodiments of a device according to the invention, the configuration of the collar may be without the frustoconical volume 28, because the gas inlets 22 are formed in the lateral wall 25 at a location situated between the opening 4 and the spraying orifice 14.

The spraying orifice dispenses the liquid according to a spray having an envelope surface. In a case in which the collar defines a frustoconical volume 29 of circular section, and in which the envelope surface of the spray is also frustoconical and of circular section, then the width of the spray and the width of the opening can be respectively measured along any axis. However, when the nozzle 10 has a spraying orifice 14 in the form of a longitudinal slot along an axis Z, such that the spray is in the form of a jet of a film along the axis Y perpendicular to this axis Z, then the width of the spray of this envelope surface and the width of the opening are measured along this axis Z. In particular, the configuration of the nozzle 10 and of the collar 20 is such that the width of the spray “Is”, measured at a distance “dr” from the spraying orifice without a collar is between 80% and 140% of the width “lo” of the opening 4 along this axis Z.

When the film extending at the outlet of the nozzle in a plane containing the axis Z, and forms there a continuous surface 15 bordered by filaments 16 and ending in drops 17, the width “If” of the continuous surface 15, FIG. 3a, measured along the axis Z at a distance equal to the distance dr by which the spraying orifice without a collar is set back is between 40% and 120% of the width of the opening 4 along this axis Z. The cumulative distance D1 and D2 may in particular represent between 40% and 120% of the set-back distance “dr”.

As can be seen in FIG. 5, the axis Z of the slot formed by the spraying orifice is not necessarily parallel to the gas dispensing axis W.

Perpendicular to the measurements of width, when the nozzle 10 has a spraying orifice 14 in the form of a longitudinal slot, a maximum thickness “Es” of the spray can be defined along an axis perpendicular to the axis Z. In this case, the opening 4 is preferably configured to have a slot shape, FIG. 7, of width “lo” and thickness “Eo”, such that the maximum thickness “Es” of the spray, measured at a distance “dr” from the spraying orifice without a collar, is between 80% and 140% of the thickness “Eo” of the opening 4 perpendicular to this axis Z.

The measurements of width and thickness of the spray are taken in the absence of the collar, since turbulence is more particularly observed in a portion corresponding to the length D2 of the spray, that in which the continuous surface of the sprayed liquid undulates, such that these turbulences locally modify the dimensions of the spray observed in the collar, and can be modulated by the flow rate of gas at the gas inlets 22 that is determined in the confinement chamber.

When the width exceeds 100% of, respectively, the width of the opening, then a phenomenon of runoff of a part of the sprayed liquid is observed along the lateral wall. A preferential proportion of the ratio between the width of the dispensing opening lo and the width of the spray Is is 95% to 120%.

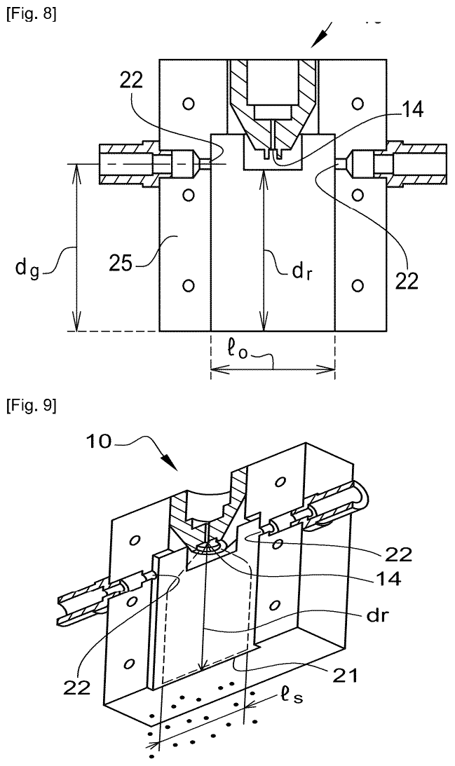

In the variant in FIGS. 7 to 9, in which the opening defines a slot and the spraying takes place via a spraying orifice provided with a slot, the gas inlets 22 are diametrically opposite one another along the axis of the slot and are situated set back from the opening 4, by a distance dg less than the distance dr by which the spraying orifice is set back relative to the opening 4.

Optionally, the reservoir 7 can also be supplied through a pipe 18 with gas provided by an ozone generator 13 so that the dispensed liquid is already enriched with dissolved ozone even before it has been sprayed and is additionally laden with dissolved ozone in the confinement chamber.

1. A spraying device for spraying ozonated water in an agricultural environment, the device comprising:
an opening for jointly dispensing liquid and gas,
a nozzle intended to be connected to a reservoir of liquid to be sprayed,
a gas inlet of a device for producing and/or storing gas,
and a collar surrounding the nozzle and the gas inlet such that an edge of this collar defines the dispensing opening of the device, and such that a spraying orifice of the nozzle is at a distance set back in the collar relative to this dispensing opening,
wherein the collar defines a chamber for confining the gas emitted through the gas inlet, the confinement chamber extending between the spraying orifice and the dispensing opening.

2. The spraying device as claimed in claim 1, wherein the nozzle has a speed of spraying of the liquid through the spraying orifice, the device for producing and/or storing gas has a speed of dispensing of the gas through the gas inlet, such that the spraying speed of the liquid is between 100 and 300 times the dispensing speed of the gas.

3. The spraying device as claimed in claim 1, wherein the spraying orifice dispenses the liquid according to a spray distributed spatially in at least one plane such that a width of the opening measured in this plane and orthogonal to a spraying axis of the nozzle represents between 80% and 140%, and more particularly between 95% and 120%, of a spray width in this same plane for a spray produced by said nozzle without its collar, this spray width being measured at a distance from the spraying orifice that is equal to the set-back distance.

4. The spraying device as claimed in claim 1, wherein the spraying orifice dispenses the liquid according to a spray distributed spatially in at least one plane and having a spray thickness transverse to this plane such that a thickness of the opening measured transverse to this plane represents more than 100%, in particular less than 140%, of the spray thickness transverse to this same plane for a spray produced by said nozzle without its collar, the spray thickness being measured at a distance from the spraying orifice that is equal to the set-back distance.

5. The spraying device as claimed claim 1, wherein the spraying orifice dispenses the liquid according to a spray having a portion in the form of liquid film, and in that a length of this film measured along a spraying axis of the nozzle represents between 40 and 120% of the set-back distance.

6. The spraying device as claimed claim 1, wherein the spraying orifice forms a longitudinal slot along an axis of elongation, and the dispensing opening has a main elongation axis parallel to the axis of the slot.

7. The spraying device as claimed in claim 1, wherein the gas inlet is set back in the collar relative to the dispensing opening, this set-back of the gas inlet possibly being greater than the set-back distance.

8. The spraying device as claimed in claim 1, wherein the spraying device has at least two gas inlets that are evenly distributed and connected to the same device for producing and/or storing gas.

9. The spraying device as claimed in claim 1, wherein the nozzle is central and the gas inlet is lateral in the collar, in particular the gas inlet is adjacent to an inner wall of the collar.

10. The spraying device as claimed in claim 1, wherein the volume of the confinement chamber, in particular between the spraying orifice and the dispensing opening, is parallelepipedal.

11. The spraying device as claimed in claim 1, wherein the spraying orifice dispenses the liquid according to a spatially distributed spray, such that the nozzle without its collar dispenses a spray having a spray volume measured in a defined volume between the spraying orifice and a distance by which this spraying orifice is set back, such that the spray volume occupies a volume less than 5% of an internal volume of the collar.

12. The spray device as claimed in claim 1, wherein the dispensing opening is a single opening, and the collar is formed by a continuous wall.

13. A method for designing a spraying device as claimed in claim 1, wherein
a nozzle is selected that is able to emit a spray of liquid distributed spatially according to an opening angle in a plane and a radial thickness transverse to this plane,
a flow rate of liquid from the nozzle is determined, in order to determine a gas flow rate as a function of this liquid flow rate,
a collar depth is determined as a function of the opening angle and the radial thickness of the spray of the nozzle.

14. An assembly for phytosanitary treatment having a spraying device as claimed in claim 1, wherein the device for producing and/or storing gas has a diversion toward the reservoir so as to enrich the liquid contained in the reservoir with gas, such that the liquid has a proportion of dissolved gas before spraying.
